(19) United States
(12) Patent Application Publication
(10) Pub. No.: US 2002/0128248 A1
(43) Pub. Date: Sep. 12, 2002

(54) SODm therapy for prevention and/or treatment of inflammatory disease

(75) Inventor: Daniela Salvemini, Chesterfield, MO (US)

Correspondence Address: SENNIGER POWERS LEAVITT AND ROEDEL, ONE METROPOLITAN SQUARE, 16TH FLOOR, ST LOUIS, MO, 63102, US

(73) Assignee: MetaPhore Pharmaceuticals, Inc

(21) Appl. No.: 09/997,974

(22) Filed: Nov. 30, 2001

Related U.S. Application Data
(63) Continuation-in-part of application No. 09/634,152, filed on Aug. 9, 2000.
Application No. 09/634,152 is a division of application No. 09/057,831, filed on Apr. 9, 1998, now Pat. No. 6,180,620.
(60) Provisional application No. 60/050,402, filed on Jun. 20, 1997.

(51) Int. Cl. A61K 31/555

(52) U.S. Cl. 514/185; 540/465

(57) ABSTRACT

The present invention relates to pharmaceutical compositions and methods using such compositions for the treatment of inflammatory disease, specifically rheumatoid arthritis. Such compositions contain a catalyst for the dismutation of superoxide which is a low molecular weight organic ligand derived metal complexes that function as mimics of the superoxide dismutase enzyme (SOD mimetics or SODms).

This application is a continuation-in-part application of co-pending U.S. application Ser. No. 09/634,152, filed Aug. 9, 2000, which is a divisional application of U.S. application Ser. No. 09/057,831, filed Apr. 9, 1998, now U.S. Pat. No. 6,180,620, issued Jan. 30, 2001, which is a non-provisional of U.S. application Serial No. 60/050,402, filed Jun. 20, 1997.

### FIELD OF THE INVENTION

The present invention relates to the use of a manganese complex of a heterocyclic pentaazacyclopentadecane ligand, which is effective as a catalyst for dismutating superoxide.

### BACKGROUND OF THE INVENTION

Inflammatory disease is any disease marked by inflammation, which is a localized protective response elicited by injury or destruction of tissues and serves to destroy, dilute, or wall off both the injurious agent and the injured tissue. Inflammation is characterized in the acute form by the classical signs of pain, heat, redness, swelling and loss of function. Inflammation occurs when, upon injury, recruited polymorphonuclear leukocytes release reactive oxygen species (“ROS”) in oxidative bursts resulting in a complex cascade of events. Histologically, it involves a complex series of events, including dilation of arterioles, capillaries, and venules, with increased permeability and blood flow; exudation of fluids, including plasma proteins; and leukocytic migration into the inflammatory focus. Inflammatory diseases include, arthritis, inflammatory bowel disease, asthma, psoriasis, lupus and other autoimmune diseases. The inflammation associated with inflammatory diseases may be caused by a multitude of inciting events, including radiant, mechanical, chemical, infectious, and immunological stimuli.

One of the most prominent inflammatory diseases is arthritis. Arthritis is a term that refers to a group of more than 100 diseases that cause joint swelling, tissue damage, stiffness, pain (both acute and chronic), and fever. Arthritis can also affect other parts of the body other than joints including but not limited to: synovium, joint space, collagen, bone, tendon, muscle and cartilage, as well as some internal organs. The two most common forms of arthritis are osteoarthritis (“OA”) and rheumatoid Arthritis (“RA”). RA is the most severe of these two forms in terms of pain; while OA is by far the most common form. RA is a systematic, inflammatory, autoimmune disease that commonly affects the joints, particularly those of the hands and feet. The onset of rheumatoid arthritis can occur slowly, ranging from a few weeks to a few months, or the condition can surface rapidly in an acute manner.

At the cellular level, inflammatory diseases are characterized by an accumulation of cytokines such as TNF-α, IL-1β, IL-6, IL-9, IL-11, IL-15, IL-5 and several belonging to the interferon family, as well as inflammatory cells (e.g. eosinophils, neutrophils, and macrophages). For arthritis specifically, these chemicals build up in the synovial fluid during an arthritic flare-up. Many of these cytokines and mediators released from inflammatory cells cause cell and tissue damage. Additionally, another significant characteristic of the inflammatory response associated with arthritis and other diseases like lupus is a process called autoimmunity. Autoimmunity occurs when T-cells mistake the body's own collagen cells as foreign antigens and set off a series of events to clear the erroneously perceived threat. This results in an attack of the body's own cells by its immune system. Autoimmunity is particularly associated with rheumatoid arthritis and lupus. The immune response associated with arthritic flare-up is also characterized by oxidative and nitrosative stress and poly ADP-ribose synthetase (“PARS”) activity. A number of strategies have been developed to suppress autoimmune diseases, most notably drugs which nonspecifically suppress the immune response.

Aspirin and related nonsteroidal anti-inflammatory drugs (“NSAIDs”) are widely used for pain and to reduce inflammation in many inflammatory diseases, but this class of compounds has inherent problems and limitations. The use of NSAIDs commonly causes stomach upset, headache, drowsiness, easy bruising, high blood pressure, and fluid retention. NSAIDs that are nonselective for the cyclooxygenase 2 (“COX-2”) enzyme produced in inflammation also inhibit constitutive cyclooxygenase 1 (“COX-1”) enzyme, causing undesirable damage to the gastric mucosa and leading to dyspepsia, gastritis, or even gastric ulcers. Gastric ulcers may cause bleeding that goes undetected and results in anemia. Furthermore, NSAIDs may affect the function of platelets, impairing the ability of blood to clot.

In moderate to advanced cases of arthritis and other inflammatory diseases, corticosteroids, gold salts, anti-malarials and systemic immunosuppressants are used. Corticosteroids are a very effective drug for the treatment of arthritis as well as other inflammatory diseases and are the most potent anti-inflammatory agents known. Therefore, corticosteroids are the most widely used anti-inflammatory drugs for both acute and chronic inflammation. For example, glucocorticoids are the most widely used immunosuppressive drugs and are pharmacologically the most potent anti-inflammatory agents known. Corticosteroids are used orally, parenterally, and frequently, intra- and peri-articularly, i.e., injections in and around joints and joint cavities. However, the side effects associated with corticosteroid use can be severe. Unfortunately the glucocorticoid side effects profile occurs at doses much lower than those required for an anti-inflammatory effect. And, because both beneficial and detrimental effects are mediated by the same glucocorticoid receptor, it is difficult to separate anti-inflammatory efficacy from fluid and electrolyte abnormalities, hypertension, hyperglycemia, increased susceptibility to infection, osteonecrosis, osteoporosis, myopathy, behavioral disturbances, cataracts, growth arrest, fat redistribution, striae, ecchymoses, acne, and hirsutism.

Rheumatoid arthritis (“RA”) is a common human autoimmune disease characterized by chronic inflammation of the synovial joints and by subsequent progressive destruction of articular tissue. Although the initiating event in RA has not yet been defined, a growing body of evidence indicates that superoxide anions (O₂⁻, the one-electron reduction product of oxygen) perpetuate the chronic inflammatory state associated with RA.

In addition to O₂⁻ reactive oxygen species (“ROS”) also include the hydroxyl radical, OH⁻, and nitric oxide, NO⁻, as well as other species. Besides RA, reactive oxygen metabolites derived from the superoxide anion are postulated to contribute to tissue pathology in a number of inflammatory diseases, such as reperfusion injury (particularly for the intestine, liver, heart and brain), inflammatory bowel disease, osteoarthritis, atherosclerosis, hypertension, cancer, skin disorders (e.g. psoriasis, dermatitis), organ transplant rejections, chemotherapy and radiation-induced side effects, pulmonary disorders (e.g. chronic obstructive pulmonary disease (“COPD”), asthma, influenza, stroke, burns, AIDs, malaria, parkinson's disease and trauma. See, for example, Simic, M. G., et al, “Oxygen Radicals in Biology and Medicine”, Basic Life Sciences, Vol. 49, Plenum Press, New York and London, 1988; Weiss J. Cell. Biochem., 1991 Suppl. 15C, 216 Abstract C110 (1991); Petkau, A., Cancer Treat. Rev. 13, 17 (1986); McCord, J. Free Radicals Biol. Med., 2, 307 (1986); and Bannister, J. V. et al, Crit. Rev. Biochem., 22, 111 (1987).

ROS are produced in vivo through normal cellular respiration and natural biological signaling and defense mechanisms. Although cellular respiration is important to maintaining life, these highly reactive byproduct molecules have been implicated in a wide range of diseases and conditions. For example, during inflammation, recruited polymorphonuclear leukocytes release ROS during the oxidative burst of phagocytosis. However, during chronic and/or systemic inflammation, the body's ability to control the levels of ROS, specifically the superoxide anion radical, becomes overwhelmed. Llesuy et al., Free Radical Biology and Medicine, 16(4), 445-451 (1994); Taylor et al., Journal of Critical Care, 10(3), 122-136 (1995). The rampant oxidative stress that occurs during this stage of sepsis quickly reduces the levels and/or activities of the body's natural antioxidants (e.g. ascorbate, superoxide dismutase, catalase, glutathione peroxidase, vitamin E) and lipid peroxides begin to accumulate. Additionally, endogenous catecholamines and cortisol may be inactivated leading to a drop in blood pressure and an increase in vascular permeability. See Macarthur et al., Inactivation of Catecholamines by Superoxide Gives New Insights on the Pathogenesis of Septic Shock, PNAS, Vol. 97, No. 17, 9753-9758 (Aug. 15, 2000).

Sources of ROS in inflammatory joints are numerous. Osteoclasts, chondrocytes, synovial cells, neutrophils/macrophages and fragmented particles of degraded extracellular matrix (which activate synovial cells and neutrophils to release ROS) are excellent sources of superoxide. Furthermore, ischemia-reperfusion takes place as the inflamed joint is used and favors the production of excess free radicals. Indeed, the mechanical function of the synovial joint distinguishes it from other tissues. It has been suggested that this mechanical activity and the continued use of an inflamed joint leads to the intermittent ischemia-reperfusion cycling which in turn results in pulses of radical activity in the joint leading to the chronicity of inflammation. As in many other organs, post-reperfusion release of O₂⁻ in the ischemic organ plays a primary role in tissue damage.

Reactive oxygen species contribute significantly to tissue injury in RA and other inflammatory diseases. See Bauerova et al., “Role of Reactive Oxygen and Nitrogen Species in Etiopathogenesis of Rheumatiod Arthritis” Gen Physiol Biophys October 1999; 18 Spec No.: 15-20. It is known, for example, that the superoxide anion is involved in the breakdown of proteins, lipids, DNA, uric acid, polysaccharides, which have been shown to be increased in rheumatiod arthritis patients. These proteins, lipids, DNA uric acid, and polysaccharides are protected from breakdown by superoxide dismutase. Also, ROS are directly involved in tissue injuries and indirectly facilitate tissue destruction by inactivating α-1-protease inhibitors that form a complex with elastase, a serine proteinase. Bauerova et al., Role of Reactive Oxygen and Nitrogen Species in Etiopathogenesis of Rheumatoid Arthritis, Gen. Physiol. Biophys. 18, Focus Issue, 15-20 (1999). Studies have shown that chondrocyte-derived ROS damage cartilage matrix and mediate matrix degradation as part of the pathogenesis of both cartilage aging and osteoarthritis. Tiku et al., Evidence Linking Chondrocyte Lipid Peroxidation to Cartilage Matrix Protein Degradation, J. Biol. Chem., Vo. 275, No. 26, 20069-20076 (Jun. 30, 2000); Mattey et al., Influence of Polymorphism in the Manganese Superoxide Dismutase Locus on Disease Outcome in Rheumatoid Arthritis, Arthritis & Rheumatism, Vol. 43, No. 4, 859-864 (April 2000).

ROS have also been implicated in the damage of hyaluronic acid (“HA”), which is depolymerised causing synovial fluid to lose its lubricating properties causing friction in the joint. Kataoka et al., Hydroxyl radical scavenging activity of nonsteroidal antiinflammatory drugs, Free Radical Res. 27, 419-427 (1997). Hyaluronan attacked by ROS yields several intermediates and end-products found in increased concentrations in the synovial fluid and serum of rheumatic patients. Orvisky et al., High-molecular-weight hyaluronan a valuable tool in testing the antioxidative activity of amphiphilic drugs stobadine and vinpocetine, J. Pharm. Biomed. Anal. 16, 419-424 (1997); Mertens, et al., Study of eosinophil-endothelial adhesion, production of oxygen radicals and release of eosinophil cationic protein by peripheral blood eosinophils of patients with rheumatoid arthritis, Clinical and Experimental Allergy, Vol. 23, 868-873 (1993). This suggests a central role for activated oxygen species derived from superoxide in the pathogenesis of rheumatoid arthritis. See, for example, Bauerova et al., Role of Reactive Oxygen and Nitrogen Species in Etiopathogenesis of Rheumatoid Arthritis, Gen. Physiol. Biophys., 18, 15-20 (1999).

Thus, it follows that one therapeutic approach to treat RA is to remove ROS. Superoxide anions are normally removed in biological systems by the formation of hydrogen peroxide and oxygen in the following reaction (hereafter referred to as dismutation):

O₂⁻+O₂⁻+2H⁺→O₂+H₂O₂.

This reaction is catalyzed in vivo by the ubiquitous superoxide dismutase enzyme (“SOD”). This reaction is the subject for which the natural superoxide dismutase enzyme or a SOD mimetic will catalyze for the purposes of this invention. Native SOD activity has been found in articular cartilage, but levels of native SOD enzyme in synovial fluids of RA patients are significantly lower than those found in normal synovial fluids. This reduced SOD activity may at least partially contribute to the pathological events associated with RA and suggests that endogenous SOD may play a role in protecting cartilage from oxidant mediated degradation. Under normal circumstances, formation of O₂⁻ is kept under tight control by endogenous superoxide dismutase (“SOD”) enzymes which include: the Mn enzyme in mitochondria (“SOD2”) and the Cu/Zn enzyme present in the cytosol (“SOD1”) and extracellular surfaces (“SOD3”). However, in acute and chronic inflammation, the production of O₂⁻ is increased at a rate that overwhelms the capacity of the endogenous SOD enzyme defense system to remove them.

An exogenous SOD, Orgotein® (bovine CuZnSOD), was used in preliminary clinical trials in patients with various inflammatory disorders including RA and osteoarthritis. Orgotein® attenuates the release of free radicals in the synovial fluid of RA patients and has shown promising results as a therapeutic in patients with rheumatoid arthritis and osteoarthritis. For instance, in patients with active classical rheumatoid arthritis affecting the knee, intra-articular injections of Orgotein ameliorated signs and symptoms as evidenced by: improved RA activity index (morning stiffness, flexion range, pain, walking time), decrease in the level of rheumatoid factor, reduced intake of rescue acetaminophen and overall improvement in physicians and patient global ratings. Clinical studies in patients with OA also revealed amelioration with respect to signs and symptoms.

Despite encouraging clinical results, Orgotein had to be removed from the market because of its origin (bovine) and the development of immune responses against Orgotein in some individuals. Other issues associated with the use of native SOD enzymes as therapeutic agents include: solution instability, bell-shaped dose response curves, high susceptibility to proteolytic digestion and limited cellular/organ penetration.

Several non-peptidic catalysts which mimic this superoxide dismutating activity have been discovered. Recently, a class of non-peptidic, low-molecular weight compounds proven to possess a comparable catalytic activity and the high selectivity of the native superoxide dismutase (“SOD”) enzymes have been reported and the use of these compounds has been suggested for assessing a better therapeutic approach in diseases mediated by superoxide overproduction (Salvemini et al., Science 8, 304-306 (1999)). A particularly effective family of non-peptidic catalysts for the dismutation of superoxide consists of the manganese(II), manganese(III), iron(II) or iron(III) complexes of nitrogen-containing fifteen-membered macrocyclic ligands which catalyze the conversion of superoxide into oxygen and hydrogen peroxide, as described in U.S. Pat. Nos. 5,874,421 and 5,637,578, all of which are incorporated herein by reference. See also, Weiss, R. H., et al., “Manganese(II)-Based Superoxide Dismutase Mimetics: Rational Drug Design of Artificial Enzymes”, Drugs of the Future 21: 383-389 (1996); and Riley, D. P., et al., “Rational Design of Synthetic Enzymes and Their Potential Utility as Human Pharmaceuticals” (1997) in CatTech, I, 41.

These mimics of superoxide dismutase have been shown to have a variety of therapeutic effects, including anti-inflammatory activity. See Weiss, R. H., et al., “Therapeutic Aspects of Manganese (II)-Based Superoxide Dismutase Mimics” In “Inorganic Chemistry in Medicine”, (Farrell, N., Ed.), Royal Society of Chemistry, in Press; Weiss, R. H., et al., “Manganese-Based Superoxide Dismutase Mimics: Design, Discovery and Pharmacologic Efficacies” (1995), In “The Oxygen Paradox” (Davies, K. J. A., and Ursini, F., Eds.) pp. 641-651, CLEUP University Press, Padova, Italy; Weiss, R. H., et al., J. Biol. Chem., 271: 26149 (1996); and Hardy, M. M., et al., J. Biol. Chem. 269: 18535-18540 (1994). Other non-peptidic catalysts which have been shown to have superoxide dismutating activity are complexes of porphyrins with iron and manganese cations.

Clinical trials and animal studies with natural, recombinant and modified superoxide dismutase enzymes have been completed or are ongoing to demonstrate the therapeutic efficacy of reducing superoxide levels in the disease states noted above. However, numerous problems have arisen with the use of the enzymes as potential therapeutic agents, including lack of oral activity, short half-lives in vivo, immunogenicity with nonhuman derived enzymes, and poor tissue distribution.

Thus, the need presently exists for effective compositions and methods for preventing and treating inflammatory disease states associated with the overproduction of ROS. Also, there is a need for compositions and methods for preventing and treating the inflammatory and non-inflammatory effects of rheumatoid arthritis associated with the overproduction of ROS.

### SUMMARY OF THE INVENTION

Other features of the present invention will be in part apparent to those skilled in the art and in part pointed out in the detailed description provided below.

The present invention provides a method for treating inflammatory disease in a subject comprising administering a therapeutically effective amount to the subject of a pentaaza-macrocyclic ligand complex catalyst represented by the following formula:

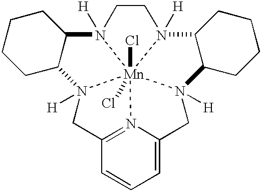

Additionally, the present invention provides a method for treatment of arthritis comprising administering a therapeutically effective amount to a subject of a pentaaza-macrocyclic ligand complex catalyst of the above formula.

The present invention further provides pharmaceutical composition for the treatment of an inflammatory disease in a subject comprising a pentaaza-macrocyclic ligand complex catalyst represented by the above formula and a pharmaceutically acceptable carrier.

### BRIEF DESCRIPTION OF THE DRAWINGS

These and other features, aspects, and advantages of the present invention will become better understood with regard to the following description, appended claims and accompanying figures where:

FIG. 1. Structure of M40403.

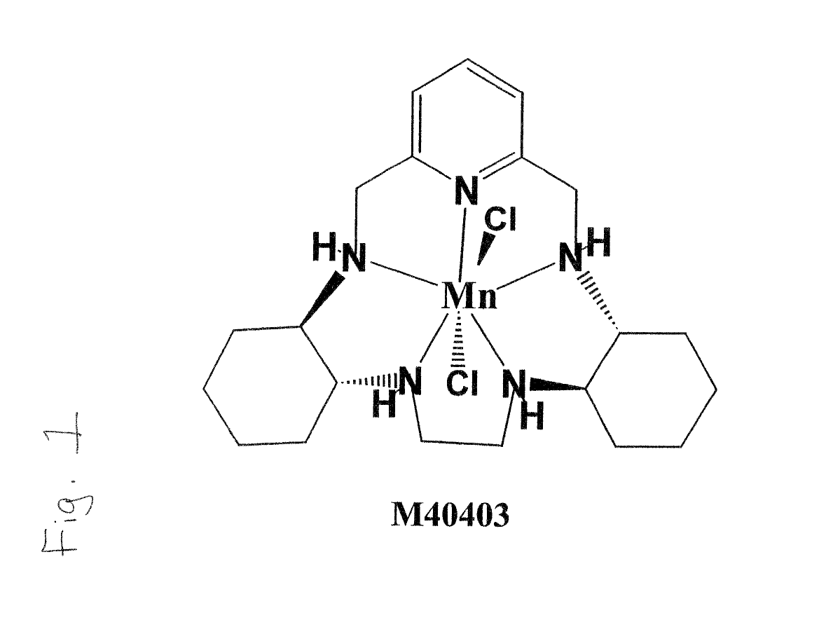

FIG. 2. Effect of M40403 on the onset of collagen-induced arthritis (“CIA”). The percentage of arthritic rats (rats showing clinical scores of arthritis >1) are represented (A). Effect of M40403 (2, −10 mg/kg i.p.) on the severity of collagen-induced arthritis. Median arthritic score during collagen-induced arthritis (B). There was a significant increase in the arthritic score from day 26 (P<0.01), and there was a significant suppression of the arthritic score way by M40403 between days 26 and 35 (P<0.01). Values are means±s.e. of 16 animals for each group. *p<0.01 versus Control. ·P<0.01 versus CIA

FIG. 3. Effect of M40403 (2-10 mg/kg i.p.) on CIA arthritis (secondary lesion). The swelling in hind paws over time (ml) was measured at 2 days intervals. Values are means±s.e. of 16 animals for each group. *p<0.01 versus Control. ·P<0.01 versus CIA

FIG. 4. Representative histology of the joint of a control animal (A), an arthritic animal (B and B1), and an M40403-treated arthritic animal (C). Note the reduction in the degree of arthritis in the joint of the rat which was treated with M40403. Original magnification: A-B-C 100×; B1 40×. Photos is representative of at least 3 experiments performed on different experimental days.

FIG. 5. Effect of M40403 treatments on histological damage score (A), and radiograph score (B). Values are means±s.e. of 16 animals for each group. *p<0.01 versus Control. ·P<0.01 versus CIA

FIG. 6. Radiographic progression of CIA in the tibiotarsal joint of rats with CIA. There is no evidence of pathology in the tibiotarsal joints of normal rats (A). The hind paws from CII-immunized (35 days) rats demonstrated bone resorption (arrow) (B). M40403 (5 mg/kg) suppressed joint pathology (arrow) and soft tissue swelling in the rat hind paw (C). Photos is representative of at least 3 experiments performed on different experimental days.

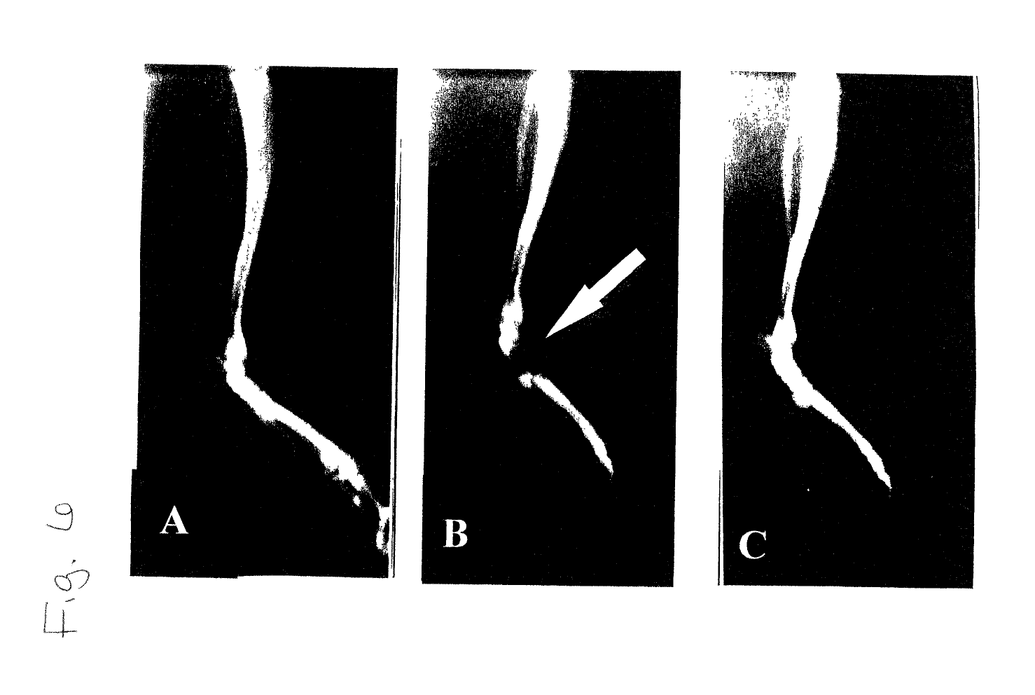

FIG. 7. Plasma levels of TNFa (A) and IL1b (B). Cytokine levels were significantly reduced in the plasma from rats which received M40403 at 5 or 10 mg/kg. The dose of 2 mg/kg only attenuated the cytokines release. Values are means±s.e. means of 16 animals for each group. *p<0.01 versus sham. ·P<0.01 versus CIA

FIG. 8. Nitrotyrosine immunostaining in the joint of a control rat (A) and the paw of a rat at 35 days of collagen-induced arthritis (B,B1). A marked increase in nitrotyrosine staining is evident in the joint in arthritis. There was a marked reduction in the immunostaining in the paw of rats which were treated with M40403 (5 mg/kg) (C). Original magnification: A-B-C 100×; B1 40×. Photos are representative of at least 3 experiments performed on different experimental days.

FIG. 9. Effect of M40403 on PARP activity: Staining was absent in control tissue (A). 35 days following collagen-induced arthritis, PAR immunoreactivity was present in the joint from CII-immunized rats (B,B1). In the paw of rats which received M40403 (5 mg/kg) (C), no positive staining was found. Original magnification: A-B-C 100×; B1 40×. Photos is representative of at least 3 experiments performed on different experimental days.

FIG. 10. Effect of M40403 on body weight gain. Beginning on day 25, the collagen-challenged rats gained significantly less weight than the normal rats, and this trend continued through day 35. M40403 (2-10 mg/kg) was able to positively affect the weight gain of CII-immunized rats. Values are means±s.e. means of 16 animals for each group. *p<0.01 versus Control. ·P<0.01 versus CIA.

FIG. 11. Anti-CII antibody titers in rats with CIA. Serum was prepared from the blood of rats (day 35) treated daily with either vehicle or M40403. Values are means±s.e. means of 16 animals for each group. *p<0.01 versus Control.

### ABBREVIATIONS AND DEFINITIONS

To facilitate understanding of the invention, a number of terms and abbreviations as used herein are defined below.

As used herein, the terms “reactive oxygen species” or “ROS” refers to a toxic superoxide anion (O₂⁻). The superoxide anion, as well as the nitric oxide (NO⁻) and the hydroxyl radical (OH⁻), are different types of free-radicals.

As used herein, the terms “non-peptidic catalysts for the dismutation of superoxide” or “non-proteinaceous catalysts for the dismutation of superoxide” mean a low-molecular weight catalyst for the conversion of superoxide anions into hydrogen peroxide and molecular oxygen. These catalysts commonly consist of an organic ligand and a chelated transition metal ion, preferably copper, manganese(II), manganese(III), iron(II) or iron(III). The term may include catalysts containing short-chain polypeptides (under 15 amino acids) or macrocyclic structures derived from amino acids, as the organic ligand. The term explicitly excludes a superoxide dismutase enzyme obtained from any species.

The term “catalyst for the dismutation of superoxide” means any catalyst for the conversion of superoxide anions into hydrogen peroxide and molecular oxygen. The term explicitly includes a superoxide dismutase enzyme obtained from any species.

The mammal patient in the methods of the invention is a mammal suffering from inflammatory disease or disorder. It is envisioned that a mammal patient to which the catalyst for the dismutation of superoxide will be administered, in the methods or compositions of the invention, will be a human. However, other mammal patients in veterinary (e.g., companion pets and large veterinary animals) and other conceivable contexts are also contemplated.

As used herein, the terms “treatment” or “treating” relate to any treatment of inflammatory disease or disorders and include: (1) preventing inflammatory disease from occurring in a subject; (2) inhibiting the progression or initiation of the inflammatory disease, i.e., arresting or limiting its development; or (3) ameliorating or relieving the symptoms of the inflammatory disease.

The term “inflammatory disease” or “inflammatory disorder” refers to any disease marked by inflammation, which may be caused by a multitude of inciting events, including radiant, mechanical, chemical, infections, and immunological stimuli. Some inflammatory diseases include, but are not limited to, arthritis, inflammatory bowel disease, asthma, psoriasis, organ transplant rejections, radiation-induced injury, cancer, lupus and other autoimmune disorders, burns, trauma, stroke, rheumatic disorders, renal diseases, allergic diseases, infectious diseases, ocular diseases, skin diseases, gastrointestinal diseases, hepatic diseases, cerebral edema, sarcoidosis, thrombocytopenia, spinal cord injury, and autoimmune disorders.

The term “arthritis” refers to inflammation of the joints and refers to a group of more than 100 rheumatic diseases that cause joint swelling, tissue damage, stiffness, pain (both acute and chronic), and fever. Arthritis can also affect other parts of the body other than joints including but not limited to: synovium, joint space, collagen, bone, tendon, muscle and cartilage, as well as some internal organs. The two most common forms of arthritis are osteoarthritis (“OA”) and rheumatoid arthritis (“RA”).

The term “therapeutically effective amounts” means those amounts that, when administered to a particular subject in view of the nature and severity of that subject's disease or condition, will have the desired therapeutic effect, e.g., an amount which will cure, or at least partially arrest or inhibit the disease or condition.

The term “joint” or “joints” refers to the place of union or junction between two or more bones of the skeleton.

All references cited herein are explicitly incorporated by reference.

### DESCRIPTION OF THE PREFERRED EMBODIMENT

The present invention is directed to methods and compositions for the prevention and treatment of inflammatory diseases comprising administering preferred compositions containing a non-proteinaceous catalyst for dismutation of superoxide. The compositions of this invention may be administered to the subject subcutaneously, intravenously, or intramuscularly. In a preferred embodiment, the compositions of this invention are administered to a subject subcutaneously or intramuscularly.

Preferably, the compound employed in the method of the present invention will comprise a non-proteinaceous catalyst for the dismutation of superoxide anions (“SOD mimic”) as opposed to a native form of the SOD enzyme. As utilized herein, the term “SOD mimic” means a low-molecular-weight catalyst for the conversion of superoxide anions into hydrogen peroxide and molecular oxygen. These catalysts consist of an organic ligand having a pentaazacyclopentadecane portion and a chelated transition metal ion, preferably manganese or iron. The term may include catalysts containing short-chain polypeptides (under 15 amino acids), or macrocyclic structures derived from amino acids, as the organic ligand. The term explicitly excludes a SOD enzyme obtained from any natural sources. SOD mimics are useful in the method of the present invention as compared to native SOD because of the limitations associated with native SOD therapies such as, solution instability, limited cellular accessibility due to their size, immunogenicity, bell-shaped dose response curves, short half-lives, costs of production, and proteolytic digestion (Salvemini et al., (1999) Science 286: 304-306). For example, the best known native SOD, CuZn, has a molecular weight of 33,000 kD. Contrastingly, the instant SOD mimics have an approximate molecular weight of 400 to 600 Daltons.

In a preferred embodiment, the SOD mimics utilized in the present invention comprise an organic ligand chelated to a metal ion. A particularly preferred catalyst is a are pentaaza-macrocyclic ligand compound, more specifically a manganese chelate of a pentaazacyclopentadecane compound.

M40403 is a stable low molecular weight, manganese-containing, non-peptidic molecule possessing the function and catalytic rate of native SOD enzymes, but with the advantage of being a much smaller molecule with a molecular weight of 483 Daltons. M40403 is not only a highly active catalyst for the dismutation of O₂⁻, but it is also highly selective for superoxide. M40403 does not react with hydrogen peroxide, nor does it directly react with other biologically relevant oxidants such as nitric oxide or peroxynitrite. M40403 is represented by the following formula:

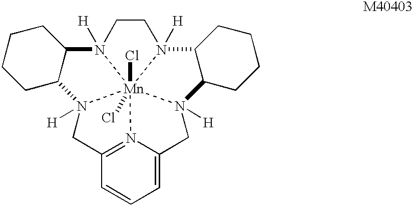

It has been discovered that M40403 is highly effective when used in the treatment of inflammatory disease in a mammal. Particularly, M40403 demonstrates effectiveness when used in the treatment of arthritis, and more particularly, in the treatment of rheumatoid arthritis. The example below presents the results of experimentation with M40403 given intraperitoneally to subjects with collagen-induced arthritis (“CIA”). CIA is a model of experimental arthritis that is induced by the injection of type II collagen (“CII”). The similarities between the joint pathology in CIA and RA suggest that CIA is a relevant animal model useful in the search for new anti-arthritic drugs. The experiment of the example below demonstrates that M40403 is highly protective in a rat model of CIA. Surprisingly, it has been discovered that protective effects of M40403 were not limited to an overall anti-inflammatory effect but included significant protection of cartilage/bone compared to untreated collagen-immunized animals, as well as inhibition of key pro-inflammatory cytokines known to be involved in the human disease.

Activity of the complexes of the present invention for catalyzing the dismutation of superoxide can be demonstrated using the stopped-flow kinetic analysis technique as described in Riley, D. P. et al., Anal. Biochem., 196: 344-349 (1991) which is incorporated herein by reference. The stopped-flow kinetic analysis is suitable for screening compounds for SOD activity or complexes of the present invention, as shown by stopped-flow analysis, correlate to treating the above disease states and disorders. However, the stopped-flow analysis is not an appropriate method for demonstrating the activity of all superoxide dismutase mimics. Other methods may be appropriate or preferred for some SOD mimics. See Weiss et al., Evaluation of Activity of Putative Superoxide Dismutase Mimics. Direct Analysis by Stopped-flow Kinetics, J.Biol.Chem. 268(31): 23049-54 (Nov. 5, 1993).

For use in treatment or prophylaxis of subjects, the compounds of the invention can be formulated as pharmaceutical or veterinary compositions. Depending on the subject to be treated, the mode of administration, and the type of treatment desired (e.g., inhibition, prevention, prophylaxis, therapy), the compounds are formulated in ways consonant with these parameters. The compositions of the present invention comprise a therapeutically or prophylactically effective dosage of a catalyst for the dismutation of superoxide in combination with at least one corticosteroid. The catalyst for the dismutation of superoxide is preferably a SOD mimetic, as described in more detail above. More preferably, the SOD mimetic is compound M40403. The SODms of this invention are preferably used in combination with a pharmaceutically acceptable carrier, either in the same formulation or in separate formulations.

The compositions of the present invention may be incorporated in conventional pharmaceutical formulations (e.g. injectable solutions) for use in treating humans or animals in need thereof. Pharmaceutical compositions can be administered by subcutaneous, intravenous, or intramuscular injection, or as large volume parenteral solutions and the like. The term parenteral as used herein includes subcutaneous injections, intravenous, intramuscular, intrasternal injection, or infusion techniques.

For example, a parenteral therapeutic composition may comprise a sterile isotonic saline solution containing between 0.1 percent and 90 percent weight to volume of the catalysts for the dismutation of superoxide. A preferred solution contains from about 5 percent to about 25 weight percent catalysts for dismutation of superoxide in solution (% weight per volume).

Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the

Total daily dose administered to a subject in single or divided doses may be in amounts, for example, from about 0.00025 to about 20 mg/kg body weight daily, more preferably from about 0.001 to about 10 mg/kg body weight daily, and more usually about 0.01 to about 3 mg/kg body weight daily, when given as a parenteral injection or continuous infusion. Dosage unit compositions may contain such amounts of sub-multiples thereof to make up the daily dose. The amount of active ingredient that may be combined with the carrier materials to produce a single dosage form will vary depending upon the subject treated and the particular mode of administration. For instance, systems such as transdermal administration or oral administration, which are substantially less efficient delivery systems, may require dosages at least an order of magnitude above those required for parenteral administration. The amount of active ingredient that may be combined with the carrier materials to produce a single dosage form will vary depending upon the host treated and the particular mode of administration. It will be appreciated that the unit content of active ingredients contained in an individual dose of each dosage form need not in itself constitute an effective amount, as the necessary effective amount could be reached by administration of a number of individual doses. The selection of dosage depends upon the dosage form utilized, the condition being treated, and the particular purpose to be achieved according to the determination of those skilled in the art.

The dosage regimen for treating a disease condition with the compounds and/or compositions of this invention is selected in accordance with a variety of factors, including the type, age, weight, sex, diet and medical condition of the patient, the route of administration, pharmacological considerations such as the activity, efficacy, pharmacokinetic and toxicology profiles of the particular compound employed, whether a drug delivery system is utilized and whether the compound is administered as part of a drug combination. Thus, the dosage regimen actually employed may vary widely and therefore may deviate from the preferred dosage regimen set forth above.

The pharmaceutical compositions of the present invention are preferably administered to a human. However, besides being useful for human treatment, these extracts are also useful for veterinary treatment of companion animals, exotic animals and farm animals, including mammals, rodents, avians, and the like. More preferred animals include horses, dogs, cats, sheep, and pigs.

The detailed description set-forth above is provided to aid those skilled in the art in practicing the present invention. Even so, this detailed description should not be construed to unduly limit the present invention as modifications and variation in the embodiments discussed herein can be made by those of ordinary skill in the art without departing from the spirit or scope of the present inventive discovery.

All publications, patents, patent applications and other references cited in this application are herein incorporated by reference in their entirety as if each individual publication, patent, patent application or other reference were specifically and individually indicated to be incorporated by reference.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

### EXAMPLE

Induction of Collagen-Induced Arthritis

Male Lewis rats (160-180 g; Charles River; Milan; Italy) were used for these studies. Collagen-induced arthritis was induced as described in Griffiths M. M. et al., Immunogenetic Control of Experimental Type II Collagen-induced Arthritis. 1. Susceptibility and Resistance among Inbred Strains of Rats, Arthritis Rheum. (2) : 781-789 (1981) and Tawara T. et al., Effects of Recombinant Human IL-1 b on Production of Prostaglandin E2, Leukotriene B4, NAG, and Superoxide by Human Synovial Cells and Chondrocytes, Inflammation (15):145-57 (1991). Bovine type II collagen (CII, Sigma) was dissolved in 0.1 M acetic acid at a concentration of 2 mg/ml by stirring overnight at 4° C. Dissolved CII was frozen at −70° C. until use. Rats were immunized with an emulsion containing 2 mg/ml of CII in Incomplete Freund's adjuvant (IFA). The emulsions were prepared by homogenizing one part CII into one part IFA (Sigma) at 4° C. On day 1, rats were injected intradermally at the base of the tail with 100 ml of the emulsion. On day 21, a second injection of CII in IFA was administered at the base of the tail.

Suppression of Collagen-Induced Arthritis by M40403

Animals were randomly divided into five groups (n=16 for each group). The first group (Group 1) was injected intraperitoneally (i.p) with vehicle only (26 mM sodium bicarbonate buffer, pH 8.1-8.3) and served as a naive group. Collagen-induced arthritis was elicited in groups 2, 3, 4 and 5. In groups 3, 4 and 5 rats were treated with M40403 at 2, 5 and 10 mg/kg respectively. M40403 was given intraperitoneally every 24 h starting from day 25. Group 2 received an equivalent volume of vehicle. Rats were evaluated daily for clinical signs of arthritis using a macroscopic scoring system which is based on redness/swelling/deformity of the joint: 0=no signs of arthritis; 1=swelling and/or redness of the paw or one digit; 2=two joints involved; 3=more than two joints involved; and 4=severe arthritis of the entire paw and digits. Arthritic index score for each rat was calculated by adding the four scores of individual paws. The Mean Arthritic Score (MAS) for each rats was calculated by dividing the total number of points scored by the group by the number of animals in the group. Clinical severity was also determined by quantitating the change in the paw volume using plethysmometry (model 7140; Ugo Basile).

Assessment of Arthritis Damage

At day 35, animals were euthanized under anaesthesia, and paws and knees were removed and fixed in 10% formalin for microscopic histological evaluation. The paws were then trimmed, placed in decalcifying solution for 24 h, embedded in paraffin, sectioned at 5 mm, stained with trichromic Van Gieson and studied using light microscopy (Dialux 22 Leitz). The following morphological criteria were considered by an investigator blinded for the treatment regime: score 0, no damage; score 1, sloughing of the articular space; score 2, inflammatory cell presence; score 3, bone erosion. Histomorphometric analysis was carried out in the proximal tibia near the joint on 5 mm thick sections, using a morphometry software, a computer with a digitizing board and a Nikon Labophot microscope equipped with both visible and UV light sources and a camera lucida attachment. Parameter for histomorphometry employed in this study, derived from Parfitt and colleagues, have been approved by an ASBMR committee. See Parfitt A. M. et al., Bone Histomorphometry: Standardization of Nomenclature, Symbols and Units, J. Bone. Miner. Res. (2):596-610 (1987). To measure bone formation, osteoblast surface was quantified relative to bone surface (Ob/Bs). To measure bone resorption, eroded surface, osteoclast surface, were quantified relative to bone surface (ES/Bs, Oc.S/Bs).

Radiography

The rats were anaesthetized with sodium pentobarbital (45 mg/kg, i.p.). Rats were placed on a radiographic box at a distance of 90 cm from the x-ray source. Radiographic analysis (Philips X12 Germany) of normal and arthritic rat hind paws was performed with a 40 kW exposure for 0.01 sec. An investigator blinded to the treatment regime scored the radiographs. The following radiographic criteria from both hind limbs were considered: score 0, no bone damage; score 1, tissue swelling and edema; score 2 , joint erosion; 3, bone erosion.

Immunohistochemical Localization of Nitrotyrosine and PARP

Tyrosine nitration, an index of the nitrosilation of proteins by peroxynitrite and/or oxygen-derived free radicals, was determined by immunohistochemistry as previously described in Cuzzocrea S. et al., Beneficial Effects of Tempol, a Membrane-permeable Radical Scavenger, in a Rodent Model of Collagen-induced Arthritis, Arthritis Rheum. (43):320-8 (2000). At day 35, the joints were trimmed, placed in decalcifying solution for 24 h, and 8 μm sections were prepared from paraffin embedded tissues. After deparaffinization, endogenous peroxidase was quenched with 0.3% H₂O₂ in 60% methanol for 30 min. The sections were permeabilized with 0.1% Triton X-100 in PBS for 20 min. Non-specific adsorption was minimized by incubating the section in 2% normal goat serum in phosphate buffered saline for 20 min. Endogenous biotin or avidin binding sites were blocked by sequential incubation for 15 min with avidin and biotin. The sections were then incubated overnight with primary anti-nitrotyrosine antibody (1:1000) or anti-poly (ADP-Ribose) (PAR) antibody (1:500) or with control solutions. Controls included buffer alone or non-specific purified rabbit IgG. Specific labelling was detected with a biotin-conjugated anti-rabbit IgG (for nitrotyrosine) or with a biotin-conjugated anti-rabbit IgG (for PARP) and avidin-biotin peroxidase complex. In order to confirm that the immunoreaction for the nitrotyrosine was specific some sections were also incubated with the primary antibody (anti-nitrotyrosine) in the presence of excess nitrotyrosine (10 mM) to verify the binding specificity. To verify the binding specificity for PAR, some sections were also incubated with only the primary antibody (no secondary) or with only the secondary antibody (no primary). In these situations, no positive staining was found in the sections indicating that the immunoreaction was positive in all the experiments carried out. All the experiments were carried out by an investigator blinded to the treatment regime.

Serum Anti-CII Antibody Determination

The serum antibodies to CII were quantitated by ELISA using biotin-labeled goat anti-rat IgG (Southern Biotechnology Associates, Inc., Birmingham, Ala.) according to the method of Watson et al., Human HLA-DRb Gene Hypervariable Region Homology in the Biobreeding BB Rat: Selection of the Diabetic-resistant Subline Response to Human Type II Collagen, J. Exp. Med. (172):1331-1339 (1990). Serum was prepared from the blood of control and treated rats 35 days post-CII immunization.

Measurement of Cytokines

TNFa and IL-1b levels were evaluated in plasma at 35 days after the induction of arthritis. The assays were carried out by ELISA using a calorimeter, commercial kits (Calbiochem-Novabiochem Corporation, USA). Each ELISA has a lower detection limit of 5 pg/ml.

Materials

Perchloric acid was obtained from Aldrich (Milan, Italy). Primary anti-nitrotyrosine antibody was from Upstate Biotech (DBA, Milan, Italy). M40403 was synthesized in house as described in Salvemini D. et al., Synzymes: Potent Non-peptidic Agents Against Superoxide-driven Tissue Injury, Science (286) :304-6 (1999). All other reagents and compounds used were obtained from Sigma Chemical Company (Sigma, Milan, Italy).

Data Analysis

All values in the figures and text are expressed as mean±standard error (s.e.m.) of the mean of n observations. For the in vivo studies, n represents the number of animals studied. In the experiments involving histology or immunohistochemistry, the photos shown are representative of at least three experiments performed on different experimental days. Data sets were examined by one- and two-way analysis of variance, and individual group means were then compared with Student's unpaired t test. For the arthritis studies, Mann-Whitney U test (two-tailed, independent) was used to compare medians of the arthritic indices. Values for the in vitro studies are presented as incidences (%), or medians. A p-value less than 0.05 was considered significant.

Results

Effect of M40403 in the Development of Collagen-Induced Arthritis

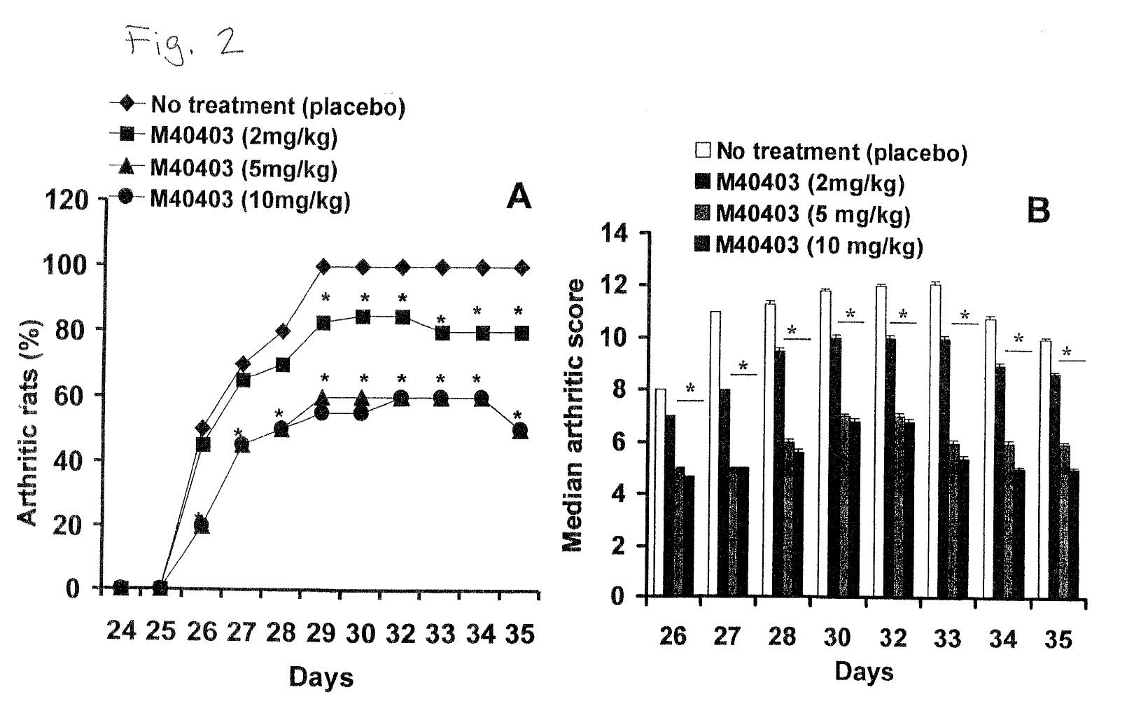

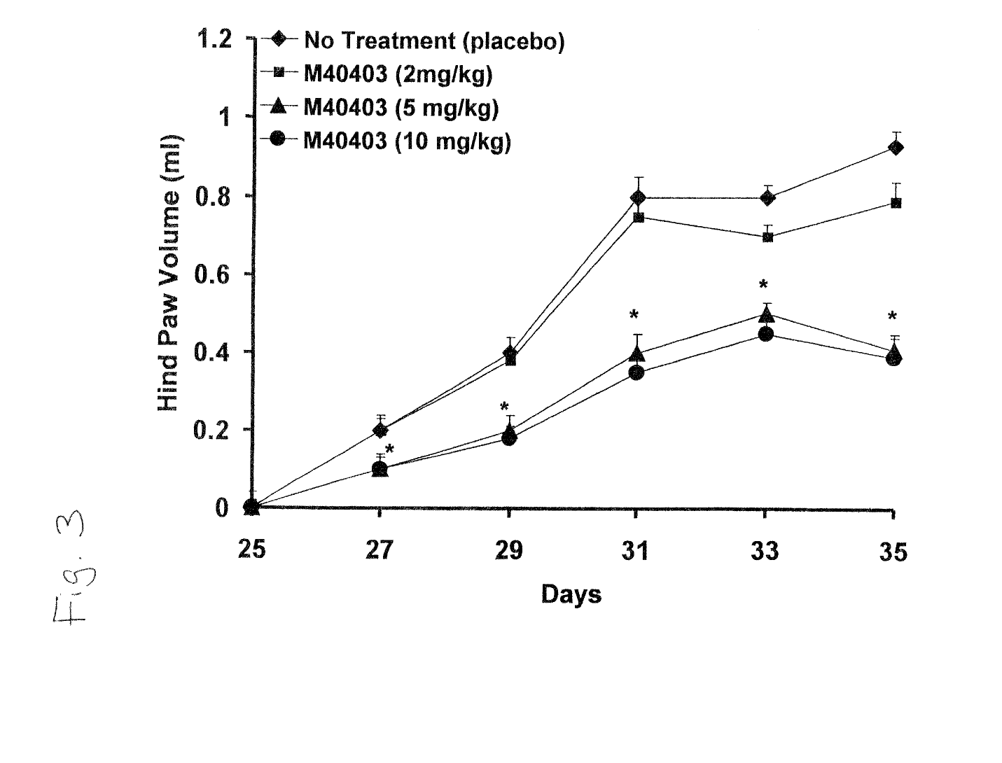

CIA developed in rats immunized with CII and clinical signs (periarticular erythema and edema) of the disease (FIG. 2A) first appeared in the hind paws between 24 and 26 after the first injection and consisted of mild erythema and swelling of the feet and ankles. Furthermore, a 100% incidence of CIA was observed by day 27 in CII-immunized rats. In contrast the maximum incidence of CIA in rats which received M40403 at 5 or 10 mg/kg starting on day 25 was 50%, (FIG. 2A) (p<0.01). No significant difference was found between the two higher doses (5 and 10 mg/kg). Hind paw erythema and swelling increased in frequency and severity in a time-dependent mode with maximum arthritis indices of approximately 13 observed between 28 and 35 days post-immunization (FIG. 2B). M40403 attenuated (P<0.01) arthritis index score as observed between days 26 and 35 post-CII immunization (FIG. 2B). The data in FIG. 3 demonstrate a time-dependent increase in hind paw volume (ml, each value represents the mean values of both hind paws) in rats immunized with CII. Maximum paw volume occurred by day 35 in the CII-immunized rats. M40403 attenuated (P<001) hind paw swelling from day 26 and 35 post-immunization, achieving a maximal response of 56% from day 28 to 35 (FIG. 3). No significant difference was found between the two higher doses (5 and 10 mg/kg).

Effects of M40403 on CIA Histopathology and Radiographic Analysis of CIA

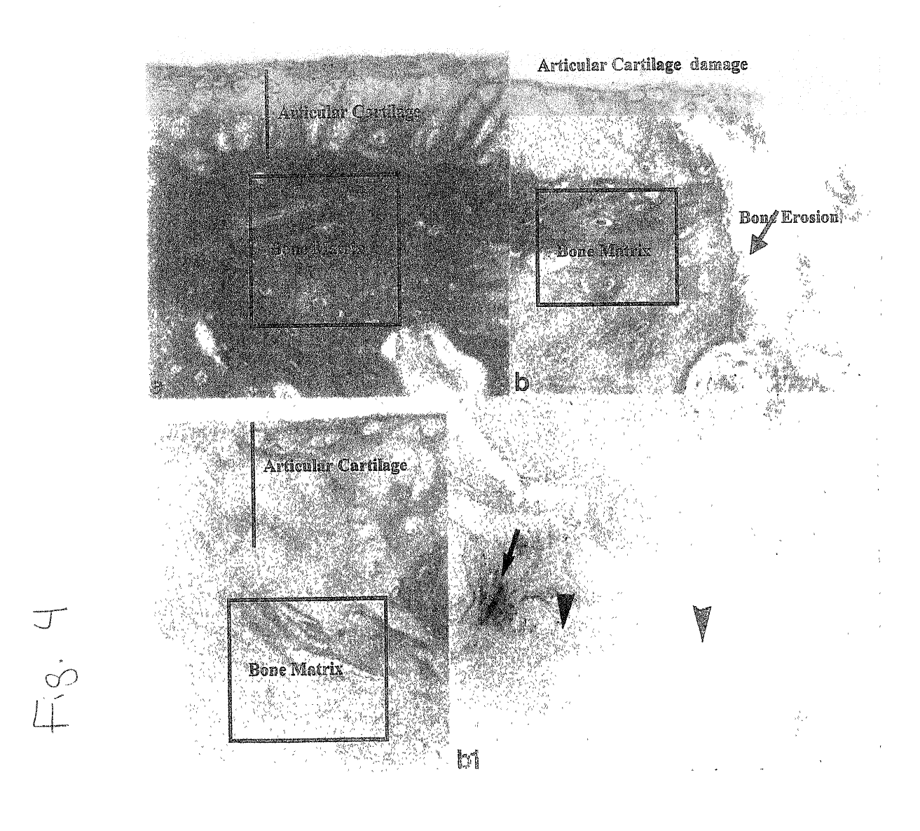

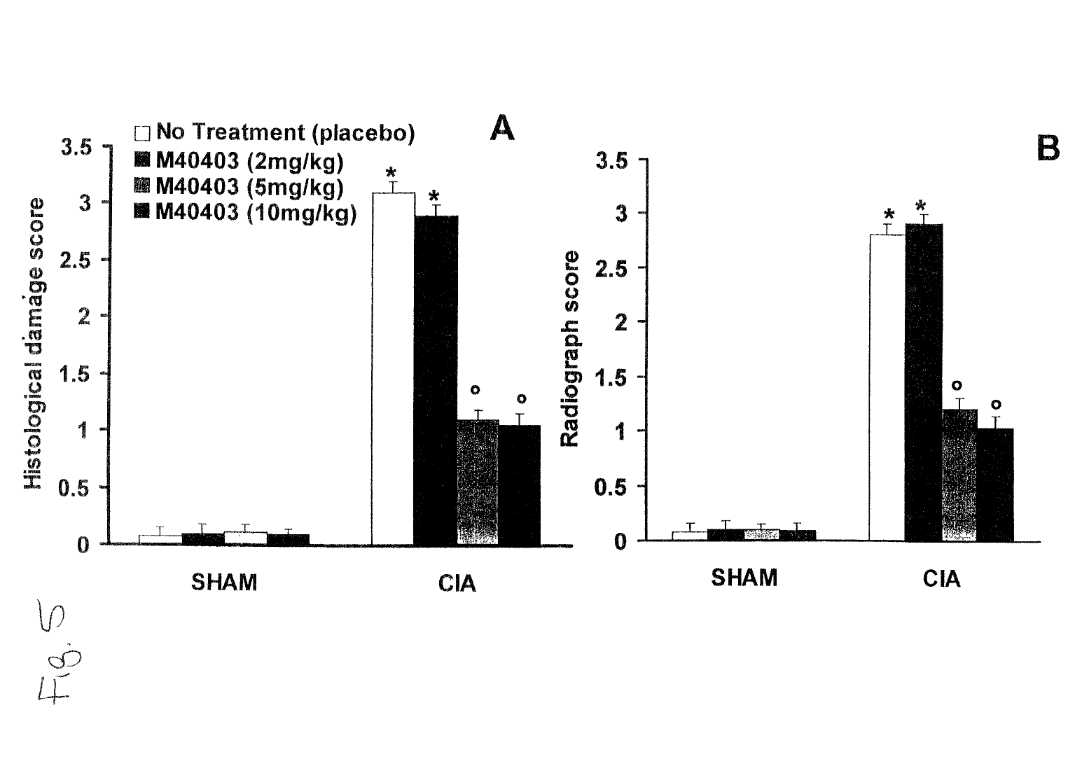

At day 35, histological evaluation of the joints in the vehicle-treated arthritic animals revealed signs of severe arthritis (FIG. 5A) characterized by articular cartilage and bone erosion (see small arrow FIG. 4B,B1, Tab 1) as well as a massive inflammatory cells infiltration (see arrow, FIG. 4B1). In the animals which received M40403 (5 mg/kg), the degree of arthritis was significantly reduced: a moderate infiltration into several of the larger joints comprised primarily of neutrophils, coupled with mild articular cartilage and bone erosion, was observed (FIG. 4C, 5A, Tab. 1). A radiographic examination of hind paws from vehicle-treated rats 35 days post CII immunization revealed bone matrix resorption (FIG. 5B, 6B) in the tibiotarsal joint. In the proximal tibia the Ob.S/Bs, the ES/Bs and Oc.S/Bs were significantly increased at 35 days after CII immunization (Tab. 1). M40403 at 5 mg/kg markedly protected against bone resorption (FIG. 5B, 6C, Tab. 1). A similar protective effect was observed in the group of animals treated with M40403 at 10 mg/kg (FIG. 5). There was no evidence of pathology in naive rats (FIG. 4A, 5A, 6A, Tab 1).

TABLE 1

| | Ob.S/BS (%) | ES/BS (%) | Oc.S/BS (%) |
|---|---|---|---|
| Sham + Vehicle | 1.21 ± 1.32 | 26.66 ± 3.32 | 1.76 ± 1.52 |
| CIA + Vehicle | 9 ± 1.02* | 40.22 ± 2.12* | 8.32 ± 1.72* |
| CIA + M40403 | 3.1 ± 0.94 | 29.98 ± 4.1° | 3.21 ± 0.99° |
| (5 mg/kg) | | | |
| CIA + M40403 | 2.9 ± 1° | 28.42 ± 3.9° | 3.41 ± 1.02° |
| (10 mg/kg) | | | |

Data are expressed as the mean value±s.e. *p<0.01 vs. sham; ·p<0.01 vs. CIA.

Key: OB.S/BS osteoblast surface; ES/BS eroded surface; Oc.S/Bs osteoclast surface.

Effect of M40403 on Cytokine Production

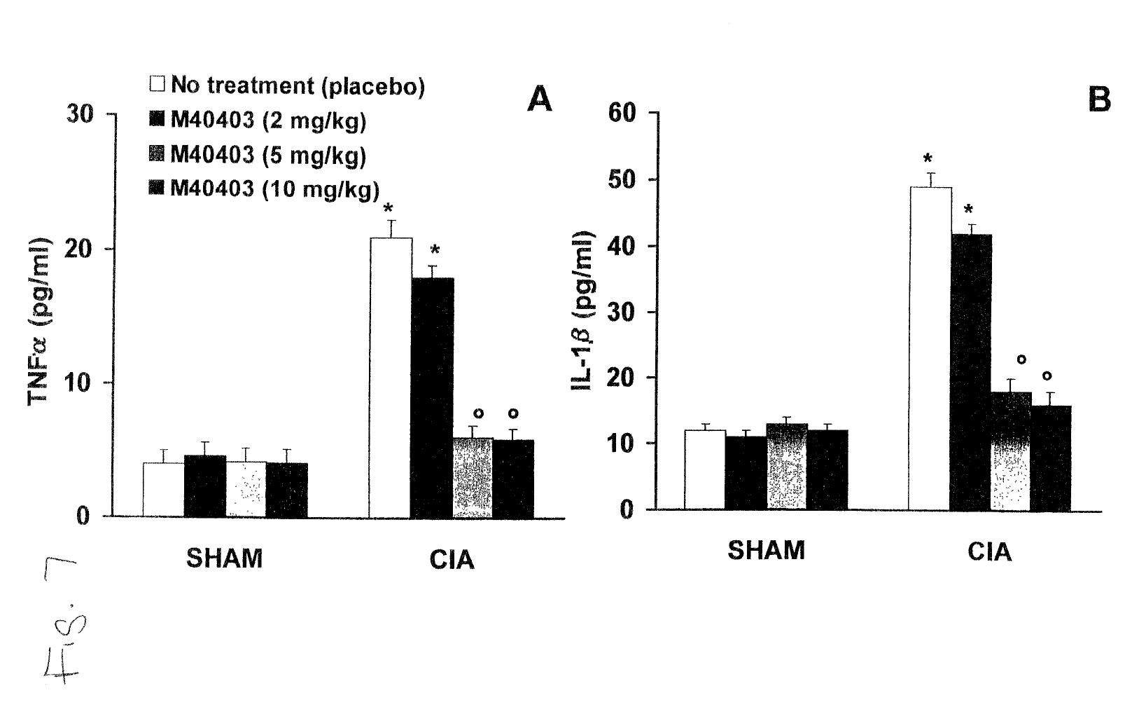

At day 35, the levels of TNFa and IL-1b were significantly elevated in the plasma of vehicle-treated CIA-immunized rats (FIG. 7). In contrast, the levels of these cytokines were significantly lower in rats which received M40403 at 5 or 10 mg/kg (FIG. 7). No significant difference was found between the two higher doses (5 and 10 mg/kg).

Nitrotyrosine Formation and PARP Activation

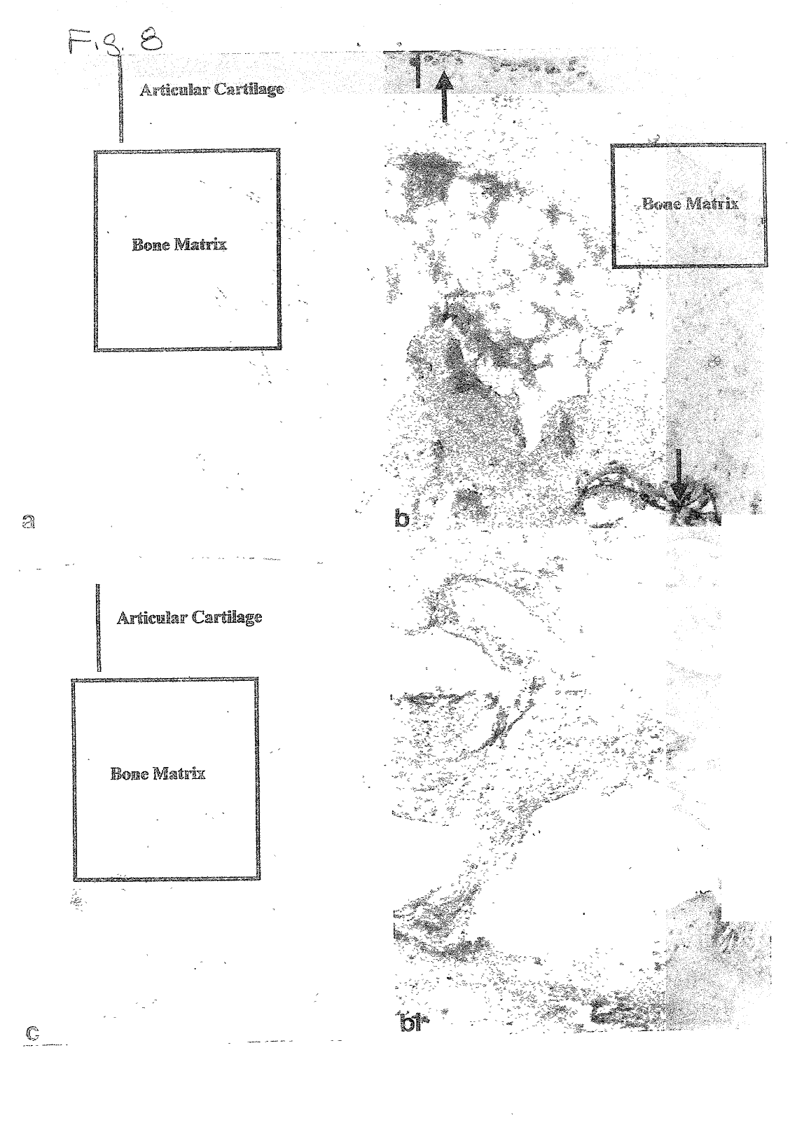

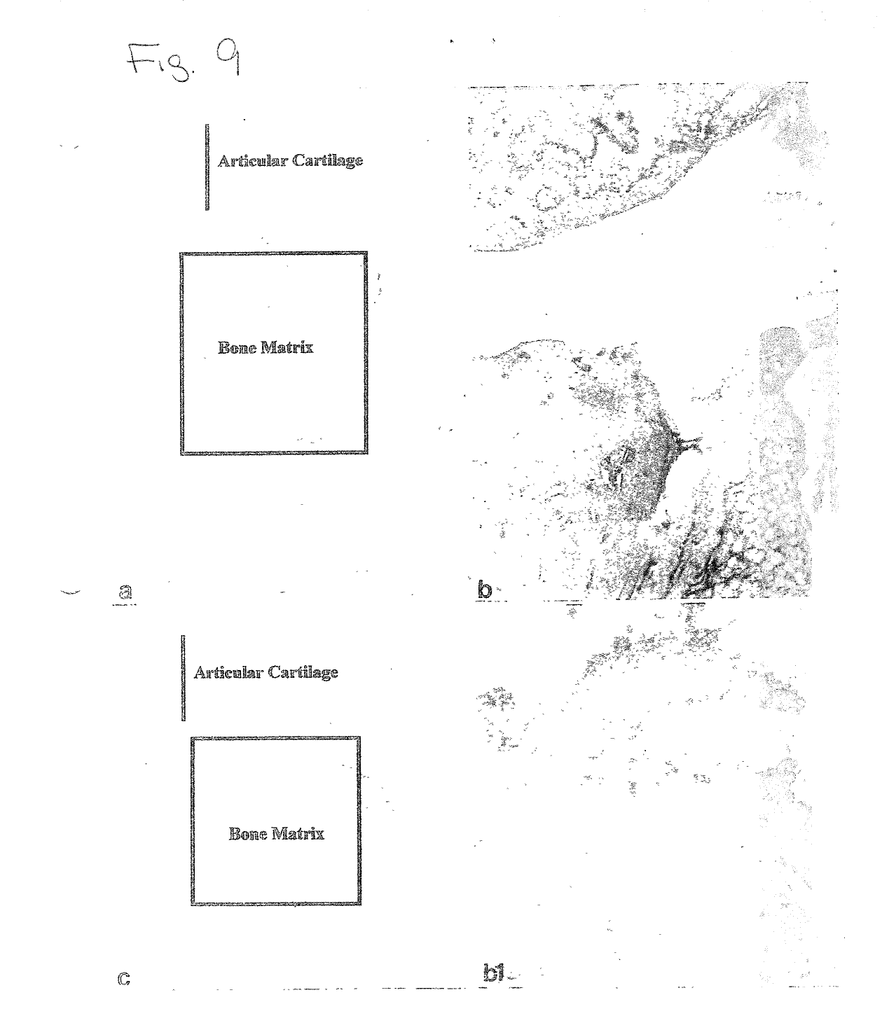

When compared to control groups (FIG. 8A), immunohistochemical analysis of joint sections obtained from vehicle-treated rats immunized with collagen type II revealed a positive staining (see arrows) for nitrotyrosine, which was primarily localized into articular cartilage and in damaged bone (FIG. 8B,B1). In contrast, no positive nitrotyrosine staining was found in the joints of CIA-immunized rats which had been treated with M40403 (5 mg/kg) (FIG. 8C). Immunohistochemical analysis of joint sections obtained from rats immunized with collagen type II also revealed a positive staining for PAR into articular cartilage and in damaged bone (FIG. 9B). In contrast, no positive staining for PAR was found in the joint of CIA-immunized rats which had been treated with M40403 (5 mg/kg) (FIG. 9C). There was no staining for either nitrotyrosine or PAR in joints obtained from naive rats (FIGS. 8A, 9A). Similar protective effect was observed in the group of animals treated with M40403 at 10 mg/kg (data not shown).

Effect of M40403 on Body Weight Gain

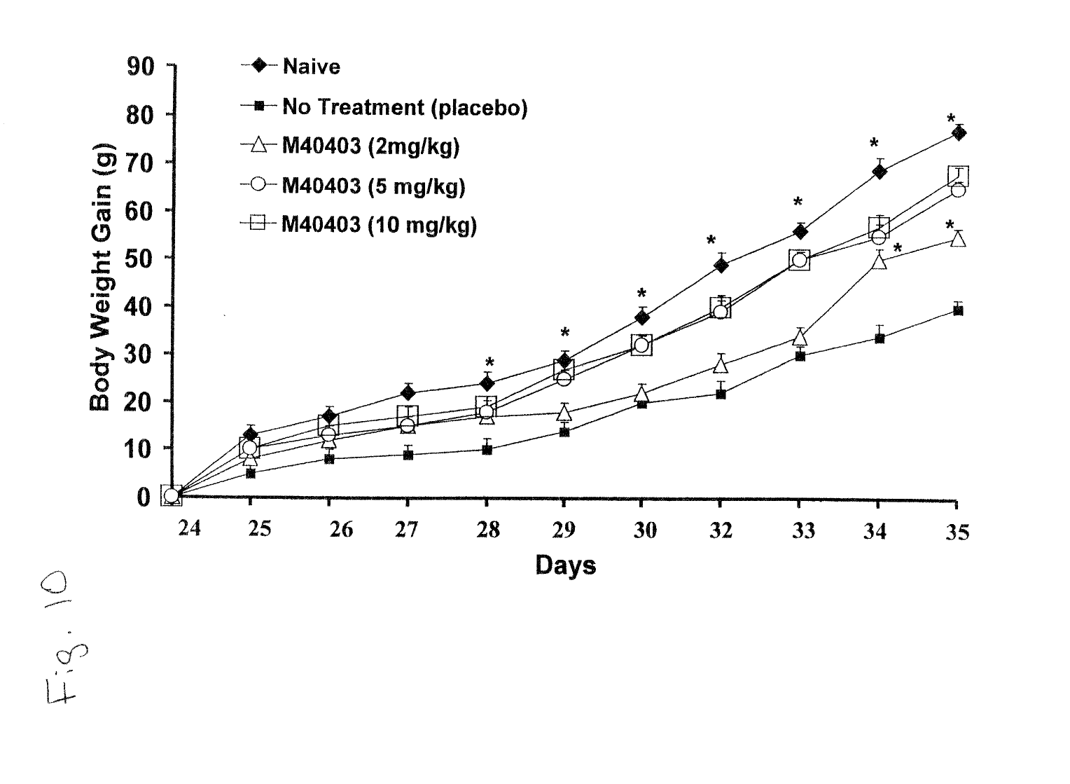

The rate and the absolute gain in body weight were comparable in naive rats and CII-immunized rats for the first week (FIG. 10). Beginning on day 25, the untreated collagen-immunized rats gained significantly less weight than the naive ones, and this trend continued through day 35. M40403 was able to positively affect in a dose dependent manner the weight gain of CII-immunized rats (FIG. 10).

Effect of M40403 on an Humoral Immunological Component of CIA

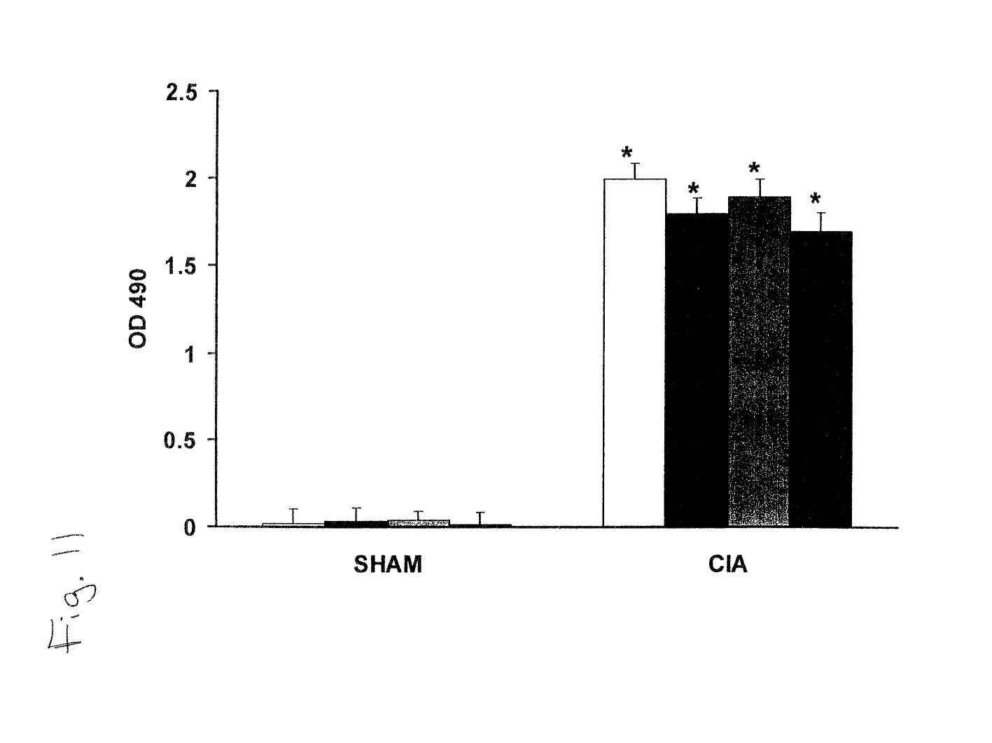

A highly significant (P<0.01) increase in serum anti-CII antibody titers was noted in CIA rats at 35 days post CII immunization (FIG. 11). M40403 had no significant effect on anti-CII antibody formation. Negligible anti-CII antibody titers were found in the serum of control rats (FIG. 11).

Discussion

Our results demonstrate that M40403 is highly protective in a rat model of collagen-induced arthritis. The protective effects of M40403 were not limited to an overall anti-inflammatory effect but included significant protection of cartilage/bone compared to untreated collagen-immunized animals, as well as inhibition of key pro-inflammatory cytokines known to be involved in the human disease.

Through both histological and radiographical evaluations, we found that M40403 was significantly protective on the cartilage and bone in tibiotarsal joints of rats immunized with CII.

Taken together, these examples indicate that O₂⁻ generated at the osteoclast-bone interface plays a role in bone matrix degradation.

Besides their key role on cartilage and bone, superoxide anions exhibit several pro-inflammatory properties. Importantly, superoxide releases (via mechanisms not yet defined) cytokines such as tumor necrosis factor-α and interleukin-1b (TNF-α and IL-1b respectively). These in turn have been implicated in the pathogenesis of RA based on the observations that anti-IL1b and anti-TNFa therapies suppress the development of CIA. These cytokines are not only pro-inflammatory but also mediate cartilage and bone destruction. A role for TNFα in the human disease has recently been shown. Thus, two anti-TNF-α therapies, Infliximab (Remicade, Centocor, Malvern, Pa.) and Etanercept (Enbrel, Immunex, Seattle, Wash.) have shown beneficial effects in patients with RA. Thus, inhibition of TNF-α is both anti-inflammatory and disease modifying. Administration of recombinant human IL-1 receptor antagonist (IL-1Ra) in patients with active RA was also found to be somewhat beneficial.

In this study, we find that the increase in TNF-α and IL-1b in the plasma of untreated rats with CIA-induced arthritis were reduced almost to basal levels in rats treated with M40403. We therefore propose that part of the beneficial anti-inflammatory and cartilage/bone protective effects of M40403 may be mediated through ROS reduction and the prevention of or inhibition of TNF-α and IL-b. This, in turn, would lead to reduced free-radical production and subsequent damage. Interestingly, IL-1b mediated cartilage matrix degradation is blocked by SOD, indicating a potential role of O₂⁻ in the IL-1b driven cartilage damage.

A predominant mechanism by which superoxide mediates its effects is through the diffusion-limited reaction with NO to generate peroxynitrite, a potent cytotoxic and pro-inflammatory molecule. Levels of nitrotyrosine, a marker of peroxynitrite formation, are elevated in synovial fluids in patients with RA consistent with a possible role for peroxynitrite, ONOO—, in human disease. Superoxide and peroxynitrite cause DNA single-strand damage, the obligatory trigger for PARP (a nuclear enzyme involved in DNA repair). Hydroxyl radical and ONOO— or peroxynitrous acid (ONOOH) also induce cellular injury partially related to the development of DNA single strand breakage. Excessive activation of PARP can rapidly deplete cellular energy stores, leading to cell death. Therefore, PARP activation is an important indicator that O₂⁻ and ONOO— are mediating cytotoxic/tissue damaging effects in acute and chronic inflammatory diseases. The role of PARP in arthritis has been shown through pharmacological and genetic manipulations. Thus, inhibitors of PARP activation such as 5-iodo-6-amino-1,2-benzopyrone were protective in a mouse model of CIA and PARP knockout mice are resistant to the development of CIA.

In the present study, significant staining for nitrotyrosine and PAR was found in the inflamed joints of untreated CII-immunized rats, and this was attenuated by M40403. These findings indicate that inhibition of peroxynitrite formation and O₂⁻/ONOO— driven PARP activation contribute to the overall protective effects of M40403 in CIA, a result consistent with the possible roles of superoxide and peroxynitrite in arthritis.

M40403 had no effect on the increase in serum anti-CII antibody titers, suggesting that it's beneficial effects are not associated with immunosuppression.

Thus, M40403 when given at the onset of the disease significantly reduced paw swelling, clinical score and the histological/radiographical severity of the disease when injected after the onset of clinical arthritis. Amelioration of joint disease was associated with near to full inhibition of TNF-α and IL-1b as well as inhibition of peroxynitrite and PARP activation, key players in RA. Thus, removal of superoxide is anti-inflammatory and results in significant protection at the level of cartilage and bone.

In view of the above, it will be seen that the several objectives of the invention are achieved and other advantageous results attained.

What is claimed is:

1. A method for treating inflammatory disease in a subject, the method comprising administering a therapeutically effective amount to the subject of a pentaaza-macrocyclic ligand complex catalyst represented by the following formula:

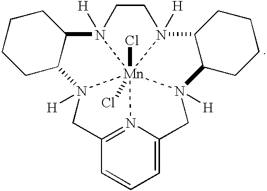

2. The method of claim 1 wherein the subject is a mammal.

3. The method of claim 2 wherein the mammal is a human.

4. A method for treatment of arthritis, the method comprising administering a therapeutically effective amount to a subject of a pentaaza-macrocyclic ligand complex catalyst represented by the following formula:

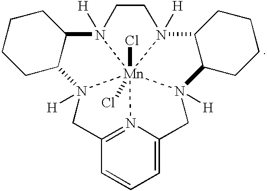

5. The method of claim 4 wherein the arthritis is rheumatoid arthritis.

6. The method of claim 4 wherein the subject is a mammal.

7. The method of claim 6 wherein the mammal is a human.

8. A pharmaceutical composition for the treatment of an inflammatory disease in a subject, the composition comprising a pharmaceutically acceptable carrier and a pentaaza-macrocyclic ligand complex catalyst represented by the following formula:

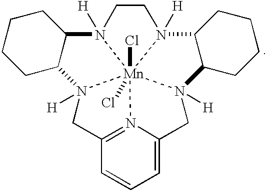

9. The composition of claim 8 wherein the inflammatory disease is arthritis.

10. The composition of claim 9 wherein the inflammatory disease is rheumatoid arthritis.
